(19) United States
(12) Patent Application Publication
(10) Pub. No.: US 2004/0266001 A1
(43) Pub. Date: Dec. 30, 2004

(54) Solid culture medium for micro-organisms and eukaryotic cells and the production method of same

(76) Inventors: Abdelkader Belardi, Bezannes (FR); Regis De Baynast, Versailles (FR); Anthony Bresin, Reims (FR)

Correspondence Address: JACOBSON HOLMAN PLLC, 400 SEVENTH STREET N.W., SUITE 600, WASHINGTON, DC, 20004, US

(21) Appl. No.: 10/494,059

(22) PCT Filed: Oct. 24, 2002
(86) PCT No.: PCT/FR02/03659

(30) Foreign Application Priority Data
Oct. 29, 2001 (FR) 0113993
Oct. 21, 2002 (FR) 0213095

(51) Int. Cl. C12N 5/02

(52) U.S. Cl. 435/404

(57) ABSTRACT

A solid culture medium which is intended, in particular, for micro-organisms and eukaryotic cells. The medium includes, by way of a gelling base, a polysaccharide having a reproduction unit including a side chain and six neutral sugars, including glucose and galactose, and an acid sugar, pyruvate substituents and acetates being present. A solution in water or a saline solution having a concentration of the polysaccharide which is greater than or equal to 2 g/l forms an elastic transparent gel.

The present invention relates to a solid culture medium for micro-organisms and eukaryotic cells and to a method for obtaining them.

Nowadays, all solid culture media contain a gelling base commonly called agar-agar. Because of the intrinsic properties of gelose media they have numerous applications in fields as diverse as basic research in molecular biology, genetic engineering, methods for identifying micro-organisms as well as the preservation thereof for maintaining collections.

However, obtaining colonies to be identified takes several days when frequently, as in the case of medical samples, time is limited.

With regard to collections of micro-organisms, the rapid depletion of the medium entails the necessity of frequent systematic subculturing.

Furthermore, one of the aims of the present invention is to provide a novel solid culture medium in which the bioavailability of the substrates is optimum.

This aim as well as others which will appear below, is achieved by a solid culture medium, characterised by the fact that it comprises, as a gelling base, a polysaccharide having a reproduction unit which has a side chain and comprises six neutral sugars, including glucose and galactose, and an acid sugar, pyruvate and acetate substituents being present, a solution in water or a saline solution having a concentration of this polysaccharide which is greater than or equal to 2 g/l forming an elastic, transparent gel.

This polysaccharide is preferably synthesised by a Rhizobium filed under the No. 1-1809 with the CNCM.

The culture medium advantageously comprises 2.2 to 6% by weight of this polysaccharide with respect to the total weight of this medium.

This solid culture medium preferably also comprises 0.5 M of sodium chloride (NaCl).

As stated above, the present invention also relates to a method for obtaining this culture medium, according to which:

a) known nutritional media are mixed in powder form;

b) added to this mixture are 2.2 to 6% by weight of the above polysaccharide in powder form;

c) the mixture thus obtained is dissolved by adding water in a sufficient quantity to meet the recommendations for the relevant medium;

d) the solution from stage c) is sterilised and,

e) the still hot sterilised product is poured as appropriate into containers.

Advantageously, at the end of stage e), it is left to rest for 24 h at ambient temperature.

If necessary, 0.5 M sodium chloride (NaCl) is preferably added to the solution from stage c).

The production example which follows and which does not have any limiting character, will allow the person skilled in the art to better understand the present invention and, in particular, its many advantages.

A culture medium is produced in accordance with the present invention from the composition of a known medium such as that known as Luria Bertani which is sold by the Difco company under the reference 0446-17-3, complemented by a heart/brain mixture sold by the OXOID company under the reference CM 225, the gelling base of which is agar-agar; however, this is replaced by a polysaccharide of the type comprising a reproduction unit which has a side chain and comprises six neutral sugars, including glucose and galactose and an acid sugar, pyruvate and acetate substituents being present; a solution in water or a saline solution of this polysaccharide having a concentration which is greater than or equal to 2 g/l forms an elastic and transparent gel. A polysaccharide of this type is also described in the French patent application published under the No. 2 759 377.

To produce this medium, nutritional media in the form of powder are mixed, then the polysaccharide in the form of powder is added in a proportion between 2.2 and 6% by weight. The powders are then taken up into solution by the addition of water in an adequate quantity to meet the recommendations for the relevant medium.

The entirety is then subjected to sterilisation, for example at 110° C. for 10 min or 121° C. for 15 min by autoclave. The various powders do not need to be perfectly taken up into solution prior to the sterilisation stage, the polysaccharide dissolving during sterilisation: a culture medium in accordance with the invention hereinafter called “inventive medium” is thus obtained.

A conventional medium containing agar-agar as the only gelling base has also been prepared and this constitutes the control medium, as well as a medium, called a mixed medium, comprising a mixture of agar-agar and polysaccharide as the gelling base. The compositions of these three culture media have been grouped together in Table 1 below.

TABLE 1

| | Control medium | Inventive medium | Mixed medium |
|---|---|---|---|
| | Concentration | Concentration | Concentration |
| Composition | in g/l | in g/l | in g/l |
| Tryptones | 10 | 10 | 10 |
| NaCl | 10 | 10 | 10 |
| Yeast extract | 5 | 5 | 5 |
| Heart/brain | 5 | 5 | 5 |
| Glucose | 2 | 2 | 2 |
| Agar-agar | 22 | 0 | 1 |
| Polysaccharide | 0 | 22 | 22 |

The media above, after sterilisation, are poured into round Petri dishes with a total surface area of 5,800 mm². After drying and a whole day of maturation, each of these media are cultured by depositing 2 μl of a starter of each of the following micro-organisms: Pseudomonas aeruginosa, Bacilis subtilis and Staphylococcus epidermidis, these being in an exponential growth phase.

After digital acquisition of the Petri dishes on which the colonies develop, the photos are printed then the colonies are cut out. Knowing that the paper has a density of 80 g/m², measuring the weight represented by the disks allows the surface of the colony to be estimated. Thus, despite the atypical form of the colonies of micro-organisms, a precise estimation of the surface of the colonies is produced. The growth of these was tracked for a period of 65 hours. The results obtained are presented in the form of curves, to which the attached drawings relate:

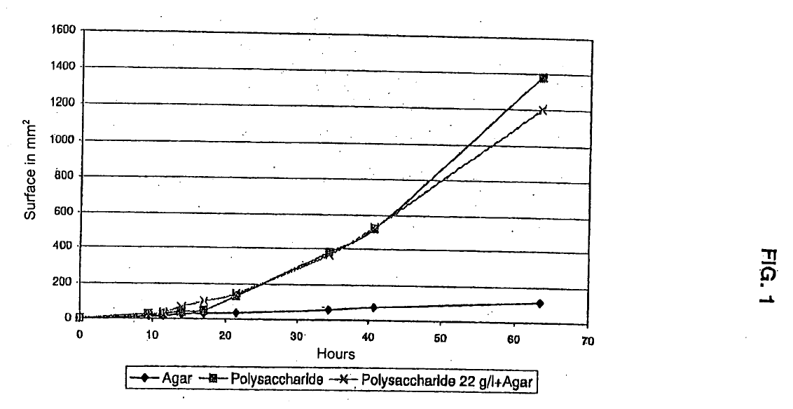

FIG. 1 shows the growth curve of Pseudomonas aeruginosa on each of the three above grown media;

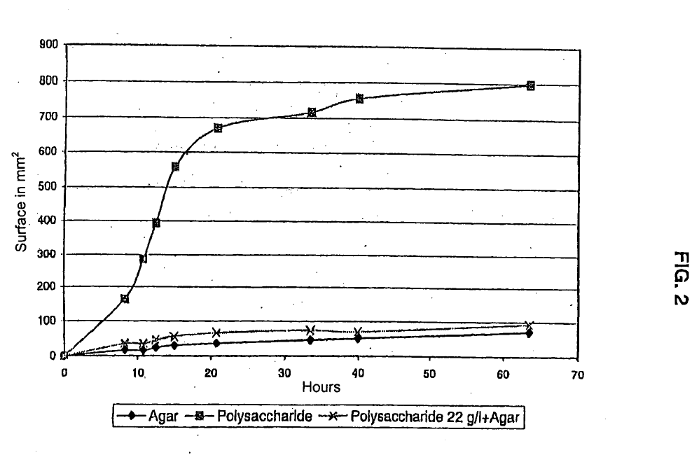

FIG. 2 shows the growth curve of Bacilis subtilis on each of the above three growth media; and,

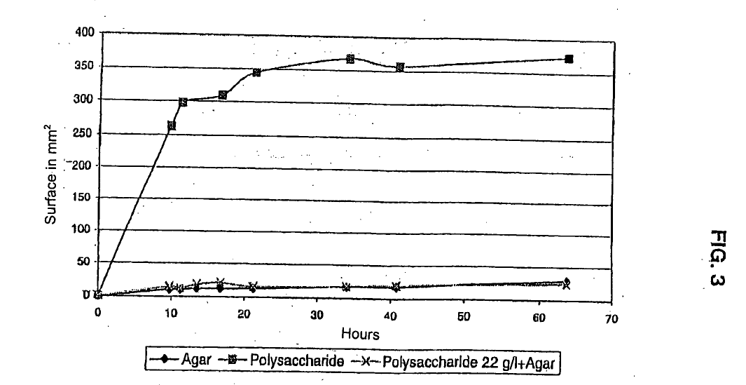

FIG. 3 shows the growth curve of Staphylococcus epidermidis on each of the three growth media above.

The culture medium in accordance with the present invention therefore allows the size of the micro-organism colonies to be increased in a very significant manner. Therefore, because of this increase in size, it is possible to identify early the micro-organisms present on the dishes.

Comparative tests with the same micro-organisms as above have also been produced, but with different proportions of polysaccharide, by adding or not adding 1 g/l of agar-agar or 0.5 M sodium chloride (NaCl). The results have been grouped together in Tables 2 and 3 below.

TABLE 2
Surface area of colonies in mm² after 63.5 hours of culture

| | Bacilus | Pseudomonas | Staphylococcus |
|---|---|---|---|
| | subtilis | aeruginosa | epidermidis |
| Agar | 72 | 115 | 29 |
| Polysaccharide | 798 | 1381 | 374 |
| Polysaccharide 22 g/l + | 755 | 1326 | 46 |
| NaCl | | | |
| Polysaccharide 22 g/l + | 93 | 1201 | 25 |
| agar | | | |
| Polysaccharide 30 g/l | 206 | 2121 | 239 |
| Polysaccharide 30 g/l + | 109 | 1379 | 38 |
| agar | | | |
| Polysaccharide 40 g/l | 179 | 2111 | 179 |
| Polysaccharide 40 g/l + | 67 | 2197 | 34 |
| agar | | | |

TABLE 3
Ratio of control surface/test surface

| | Bacilus | Pseudomonas | Staphylococcus |
|---|---|---|---|
| | subtilis | aeruginosa | epidermidis |
| Agar | 1 | 1 | 1 |
| Polysaccharide | 11 | 12 | 13 |
| Polysaccharide 22 g/l + | 11 | 12 | 2 |
| NaCl | | | |
| Polysaccharide 22 g/l + | 1 | 10 | 1 |
| agar | | | |
| Polysaccharide 30 g/l | 3 | 18 | 8 |
| Polysaccharide 30 g/l + | 2 | 12 | 1 |
| agar | | | |
| Polysaccharide 40 g/l | 2 | 18 | 6 |
| Polysaccharide 40 g/l + | 1 | 19 | 1 |
| agar | | | |

It appears from the different tests that the growth concentration of polysaccharide or the addition of agar-agar at 1 g/l tends to limit the difference between the control and the medium comprising the polysaccharide at 22 g/l. Nevertheless, it appears that the use of the polysaccharide favours a rapid colonisation of the surface of the culture medium whatever the concentration compared to the control medium.

It will not escape the person skilled in the art that, advantageously, this novel culture medium, because of the use of the polysaccharide described in the document FR-2 759 377-A favours the optimum bioavailability of this substrate while facilitating the diffusion of the materials in the matrix of the culture medium. The micro-organisms being cultured on this gel just as easily access carbonaceous and nitrogenous substrates and growth factors; therefore they continue their growth while the medium only containing agar-agar stops the growth because of exhaustion of the nutrients at the periphery of the colony.

The accessibility to the substrate and water which results from the ability of the polysaccharide described in the document FR-2 759 377-A to collect liquids, allows the affinity between the micro-organism cells and the surface of the gelose medium to be significantly reduced. This reduction in the tension between the micro-organisms allows an easier spreading of the colonies and, therefore, a more rapid colonisation of the surface and hence the creation of bio-films.

The increase in the speed of growth of the micro-organisms thus allows the early detection and identification or otherwise of pathogenic micro-organisms during medical analyses and therefore allows an earlier targeted treatment to be defined.

Moreover, it is possible to obtain colonies of genetically modified micro-organisms clearly superior in size to those obtained on the conventionally used culture medium. In fact, on the latter, owing to the fragility and the small number of such strains and the rapid exhaustion of the nutrients at the periphery of the micro-organisms, the colonies obtained are difficult to detect.

Equally the culture medium which is the subject of the present invention, is particularly suitable for preserving collections of micro-organisms. In fact, on conventional culture media, the micro-organsims rapidly turn into a resistant form because of the exhaustion of the readily available nutrients. It is therefore necessary to make frequent subculturings of the colonies being cultured to preserve the strains in vegetative form.

In the case of the culture in accordance with present invention, the increase in the availability of the nutrients means that the micro-organisms cultivated on this gel remain in a vegetative form much longer, allowing the subculturing of the colonies to become considerably less frequent.

Equally, the polysaccharide used to make the culture medium has a substantial liquid retention capacity and this allows the formation of condensation on the cover of the Petri dish during incubation to be avoided. Thus, the observation of the colonies being cultured is facilitated, all the more so when the medium containing the polymer is transparent. Moreover, the risks of contamination are reduced, since it is no longer necessary to open the dish and remove the condensation prior to observation.

It goes without saying that the use of this medium is particularly suitable for tests for identifying the respiratory modes of micro-organisms, in other words:

obligate aerobic,

facultative (aerobic), (anaerobic); or

obligate anaerobic.

For example, the inoculation of Saccharomyces cerevisae (facultative anaerobic, aerobic micro-organism) and of Botrytis (obligate aerobic micro-organism) on a medium with a meat/liver base containing the polysaccharide described in the document FR-2 759 377-A, allows the respiratory characters of these micro-organisms to be confirmed with a shorter incubation time than on a conventional culture medium.

Moreover, certain micro-organisms, on culture media containing polysaccharide described in the document FR-2 759 377-A form colonies with characteristic forms and/or colours. Thus the identification tests are facilitated by a prior macroscopic observation. The taxonomy of the species studied is therefore easier.

Finally the polysaccharidic nature of the molecule described in the document FR-2 759 377-A and, in particular, the presence of a molecule of pyruvate, acetate, glucose and galactose in its structure, makes of this polysaccharide contained in the culture medium which is the subject of the present invention, a carbonaceous substrate which can be used by micro-organisms possessing the hydrolysis enzymes of this substrate.

1. A solid culture medium, characterised by the fact that it comprises, as a gelling base, 2.2 to 6% by weight of this polysaccharide with respect to the total weight of said medium, of a polysaccharide having a reproduction unit which has a side chain and comprises six neutral sugars, including glucose and galactose, and an acid sugar, pyruvate and acetate substituents being present, a solution in water or a saline solution having a concentration of this polysaccharide which is greater than or equal to 2 g/l forming an elastic, transparent gel.

2. A culture medium according to claim 1, characterised by the fact that the polysaccharide is synthesised by a Rhizobium registered under the No. 1-1809 with the CNCM.

3. A culture medium according to claim 1, characterised by the fact that it also comprises 0.5 M sodium chloride.

4. A method for obtaining a solid culture medium for micro-organisms and eukaryotic cells according to claim 1, characterised in that it comprises the following steps:
a) media are mixed in powder form;
b) added to this mixture are 2.2 to 6% by weight of a polysaccharide having a reproduction unit which has a side chain and comprises six neutral sugars, including glucose and galactose, and an acid sugar, pyruvate and acetate substituents being present, a solution in water or a saline solution having a concentration of this polysaccharide which is greater than or equal to 2 g/l forming an elastic, transparent gel;
c) the mixture thus obtained is dissolved;
d) sterilisation takes place for 10 minutes at 110° C.; and,
e) the still hot sterilised product is poured as appropriate into containers.

5. A method according to claim 4, characterised by the fact that at the end of stage e), it is allowed to rest for 24 hours at ambient temperature.

6. A method according to claim 4, characterised by the fact that 0.5 M sodium chloride (NaCl) is added to the solution from stage c).
